(19) United States
(12) Patent Application Publication
(10) Pub. No.: US 2012/0015977 A1
(43) Pub. Date: Jan. 19, 2012

(54) QUINOLINONE DERIVATIVES USEFUL FOR THE TREATMENT OF CNS DISORDERS

(75) Inventors: Antonio Nardi, Herzogenrath (DE); Helle Kirstein Erichsen, Vaerlose (DK); Karin de Linde Troelsen, Vaerlose (DK)

(73) Assignee: NEUROSEARCH A/S, Ballerup (DK)

(21) Appl. No.: 13/144,885

(22) PCT Filed: Jan. 18, 2010
(86) PCT No.: PCT/EP2010/050515

§ 371 Date: Sep. 21, 2011

(30) Foreign Application Priority Data
Jan. 19, 2009 (DK) PA 2009 00073

(51) Int. Cl. A61K 31/4704; A61P 25/00; A61P 25/08; A61P 25/16; A61P 25/22; A61P 25/24; A61P 25/18; A61P 25/14; A61P 29/00; A61P 9/06; A61P 1/04; A61P 1/12; A61P 1/00; A61P 9/12; A61P 17/10; A61P 17/00; A61P 25/28; C07D 215/227

(52) U.S. Cl. 514/312; 546/157

(57) ABSTRACT

The present invention relates to novel quinolinone derivatives (I), efficacious in animal models of CNS disorders and, as such, valuable candidates for the prevention or treatment of CNS (Central Nervous System) diseases or disorders. In other aspects the invention relates to pharmaceutical compositions comprising the quinolinone derivatives of the invention and to the use of these compounds for therapeutic applications.

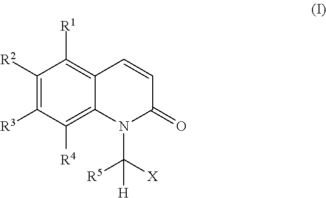

### TECHNICAL FIELD

The present invention encompasses novel quinolinone derivatives, which are surprisingly found to be efficacious in animal models of CNS disorders and, as such, are valuable candidates for the prevention or treatment of CNS (Central Nervous System) diseases or disorders.

### BACKGROUND ART

Epilepsy affects 0.5-1.0% of the population and it is a common neurological disorder characterized by recurrent spontaneous seizures. Although a number of antiepileptic drugs are available it is currently estimated that nearly 30% of epilepsy patients remain inadequately treated with available drugs. In addition, many of the antiepileptic drugs cause unpleasant adverse effects. Thus, there is a clear need to identify new antiepileptics for use in the treatment-resistant patients.

Essential tremor is one of the most common movement disorders affecting 3-4% of the population. Because of the high prevalence of the disorder, and the limited treatment exploring possible new treatments is of high interest. One of the most common animal models for essential tremor is the harmaline induced tremor. Harmaline induces a generalized tremor by acting on the inferior olivary system—the same system that in patients suffering from essential tremor, shows an increased activity (Paterson et al, Eur J Pharmacol 2009 616(1-3); 73-80)

WO 2008/132139 and WO 2008/132142 describe certain heterocyclic derivatives useful for the treatment of various CNS disorders including epilepsy.

### SUMMARY OF THE INVENTION

The present invention is devoted to the provision of novel quinolinone derivatives which have surprisingly found to be efficacious in animal models of CNS disorders.

A further object is the provision of compounds with more optimal pharmacodynamic and/or pharmacokinetic properties such as kinetic behaviour, bioavailability, solubility, efficacy and/or adverse effects.

In its first aspect the invention provides novel quinolinone derivative represented by Formula I

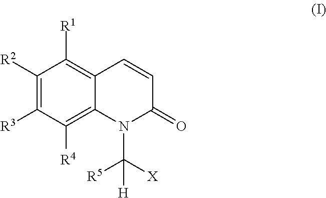

a stereoisomer thereof or a mixture of its stereoisomers, or a pharmaceutically acceptable salt thereof, wherein

R¹, R², R³ and R⁴, independently of each other, represent hydrogen, halo, cyano or SO₂CH₃;

R⁵ represents hydrogen, halo or alkyl; and

X represents an imidazole group, or the group —(CO)N(R⁶, R⁷), wherein R⁶ and R⁷, independently of each other, represent hydrogen or alkyl.

In a second aspect the invention provides pharmaceutical compositions comprising a therapeutically effective amount of the quinolinone derivative of the invention, or a pharmaceutically acceptable addition salt thereof, together with at least one pharmaceutically acceptable carrier or diluent.

Viewed from another aspect the invention relates to the use of the quinolinone derivative of the invention, or a pharmaceutically acceptable addition salt thereof, for the manufacture of pharmaceutical compositions/medicaments for the treatment, prevention or alleviation of a disease or a disorder or a condition of a mammal, including a human, which disease, disorder or condition is responsive to anticonvulsant drugs.

In yet another aspect the invention provides a method for treatment, prevention or alleviation of diseases, disorders or conditions of a living animal body, including a human, which disorder, disease or condition is responsive to modulation of CNS, and which method comprises the step of administering to such a living animal body in need thereof a therapeutically effective amount of the quinolinone derivative of the invention. Other objects of the invention will be apparent to the person skilled in the art from the following detailed description and examples.

### DETAILED DISCLOSURE OF THE INVENTION

### Quinolinone Derivatives

In its first aspect the invention provides novel quinolinone derivative represented by Formula I

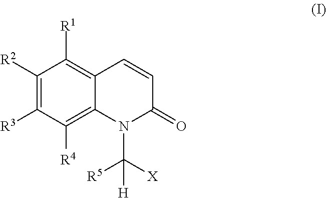

a stereoisomer thereof or a mixture of its stereoisomers, or a pharmaceutically acceptable salt thereof, wherein

R¹, R², R³ and R⁴, independently of each other, represent hydrogen, halo, cyano or SO₂CH₃;

R⁵ represents hydrogen, halo or alkyl; and

X represents an imidazole group, or the group —(CO)N(R⁶, R⁷), wherein R⁶ and R⁷, independently of each other, represent hydrogen or alkyl.

In a preferred embodiment the quinolinone derivative of the invention is a compound of Formula I, a stereoisomer thereof or a mixture of its stereoisomers, or a pharmaceutically acceptable salt thereof, wherein R¹, R², R³ and R⁴, independently of each other, represent hydrogen, halo, cyano or SO₂CH₃.

In a more preferred embodiment R¹, R², R³ and R⁴, independently of each other, represent hydrogen or halo, and in particular bromo.

In another more preferred embodiment one of R¹, R², R³ and R⁴ represents halo, cyano or SO₂CH₃; and the other three of R¹, R², R³ and R⁴ represent hydrogen.

In a third more preferred embodiment one of R¹, R², R³ and R⁴ represents halo, and in particular bromo; and the other three of R¹, R², R³ and R⁴ represent hydrogen. In a special embodiment, R² represents bromo; and R¹, R³ and R⁴ represent hydrogen. In a further embodiment, R² represents chloro; and R¹, R³ and R⁴ represent hydrogen. In a still further embodiment, R⁴ represents bromo; and R¹, R² and R³ represent hydrogen. In a further embodiment, R⁴ represents chloro; and R¹, R² and R³ represent hydrogen.

In a fourth more preferred embodiment two of R¹, R², R³ and R⁴ represents halo, cyano or SO₂CH₃; and the other two of R¹, R², R³ and R⁴ represent hydrogen.

In a fifth more preferred embodiment two of R¹, R², R³ and R⁴ represents halo; and the other two of R¹, R², R³ and R⁴ represent hydrogen.

In another preferred embodiment the quinolinone derivative of the invention is a compound of Formula I, a stereoisomer thereof or a mixture of its stereoisomers, or a pharmaceutically acceptable salt thereof, wherein R⁵ represents hydrogen, halo or alkyl.

In a more preferred embodiment R⁵ represents hydrogen.

In another more preferred embodiment R⁵ represents halo.

In a third more preferred embodiment R⁵ represents alkyl. In a special embodiment, R⁵ represents ethyl.

In a third preferred embodiment the quinolinone derivative of the invention is a compound of Formula I, a stereoisomer thereof or a mixture of its stereoisomers, or a pharmaceutically acceptable salt thereof, wherein X represents an imidazole group, or the group —(CO)N(R⁶, R⁷), wherein R⁶ and R⁷, independently of each other, represent hydrogen or alkyl.

In a more preferred embodiment X represents an imidazole group.

In another more preferred embodiment X represents an imidazole group selected from imidazol-1-yl and 1H-imidazol-4-yl.

In a third more preferred embodiment X represents an imidazol-1-yl group.

In a fourth more preferred embodiment X represents a 1H-imidazol-4-yl group.

In a fifth more preferred embodiment X represents —(CO)N(R⁶, R⁷), wherein R⁶ and R⁷, independently of each other, represent hydrogen or alkyl.

In a sixth more preferred embodiment X represents —(CO)NH₂.

In a seventh more preferred embodiment X represents —(CO)NH(CH₃).

In an eight more preferred embodiment X represents —(CO)N(CH₃)₂.

In a yet further embodiment the quinolinone derivative of the invention is a compound of Formula I, a stereoisomer thereof or a mixture of its stereoisomers, or a pharmaceutically acceptable salt thereof, wherein:

one of R² and R⁴ represents halo, such as bromo or chloro; and the other three of R¹, R², R³ and R⁴ represent hydrogen;

R⁵ represents hydrogen or alkyl;

X represents —(CO)N(R⁶, R⁷), wherein R⁶ and R⁷, independently of each other, represent hydrogen or alkyl.

In a most preferred embodiment the quinolinone derivative of the invention is
2-(6-Bromo-2-oxo-1-quinolyl)acetamide;
2-(6-bromo-2-oxo-1-quinolyl)butanamide;
2-(8-bromo-2-oxo-1-quinolyl)acetamide;
2-(6-chloro-2-oxo-1-quinolyl)acetamide;
2-(8-chloro-2-oxo-1-quinolyl)acetamide;
2-(8-chloro-2-oxo-1-quinolyl)butanamide;
2-(6-chloro-2-oxo-1-quinolyl)butanamide;
2-(8-bromo-2-oxo-1-quinolyl)butanamide;

a stereoisomer thereof or a mixture of its stereoisomers, or a pharmaceutically acceptable salt thereof.

Any combination of two or more of the embodiments described herein is considered within the scope of the present invention.

### Definition of Substituents

In the context of this invention halo represents fluoro, chloro, bromo or iodo.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

### Pharmaceutically Acceptable Salts

The quinolinone derivative of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a quinolinone derivative of the invention include alkali metal salts, such as the sodium salt of a compound of the invention containing a carboxy group.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention, including compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Steric Isomers

It will be appreciated by those skilled in the art that the quinolinone derivatives of the present invention may exist in different stereo isomeric forms, including enantiomers, diastereomers, as well as geometric isomers (cis-trans isomers). The invention includes all such stereoisomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optically active starting materials or intermediates.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Producing Quinolinone Derivatives

The quinolinone derivative of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The animal models being used for the discovery of novel antiepileptic drugs are numerous. Among these, a low frequency (6 Hz) electroshock seizure model has recently come into the limelight as a potential model of pharmacoresistant epilepsy (see Epilepsy: a comprehensive textbook, pag. 1477, by Jerome Engel, Timothy A. Pedley, Jean Aicardi, Marc A Dichter, Solomon Moshé—2007—Medical), and it is therefore an animal model of primary interest in antiepileptic drug discovery. The compounds of the invention have shown remarkable activity to protect against 6 Hz seizures.

The present invention is devoted to the provision of novel agents that are valuable candidates for the prevention or treatment of CNS diseases or disorders. The compounds of the invention may also be useful as diagnostic tools or monitoring agents in various diagnostic methods, and in particular for in vivo receptor imaging (neuroimaging), and they may be used in labelled or unlabelled form.

In a preferred embodiment the disease, disorder or condition contemplated according to the invention is epilepsy, epileptogenesis, convulsions, seizure disorders, tremor, essential tremor, myoclonus, anxiety, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, convulsions, diarrhoea, asthma, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to postherpetic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms including heroin, cocaine and morphine, benzodiazepine withdrawal symptoms including benzodiazepine-like drugs and alcohol.

In a more preferred embodiment the disease, disorder or condition is a epilepsy, epileptogenesis, convulsions, seizure disorders, tremor, essential tremor, myoclonus, anxiety, Parkinson's disease, tardive dyskinesia, hyperkinesia.

In another more preferred embodiment the disease, disorder or condition is a epilepsy, epileptogenesis, convulsions and seizure disorders.

In a third more preferred embodiment the disease, disorder or condition is pain, including mild, moderate or even severe pain of acute, chronic or recurrent character, as well as pain caused by migraine, postoperative pain, and phantom limb pain. The pain may in particular be neuropathic pain, chronic headache, central pain, pain related to diabetic neuropathy, to postherpetic neuralgia, or to peripheral nerve injury.

Finally the compounds of the invention may be useful for the treatment of withdrawal symptoms caused by termination of use of addictive substances. Such addictive substances include nicotine containing products such as tobacco, opioids such as heroin, cocaine and morphine, benzodiazepines and benzodiazepine-like drugs, and alcohol. Withdrawal from addictive substances is in general a traumatic experience characterised by anxiety and frustration, anger, anxiety, difficulties in concentrating, restlessness, decreased heart rate and increased appetite and weight gain.

In this context “treatment” covers treatment, prevention, prophylactics and alleviation of withdrawal symptoms and abstinence as well as treatment resulting in a voluntary diminished intake of the addictive substance.

### Pharmaceutical Compositions

In another aspect the invention provides novel pharmaceutical compositions comprising a therapeutically effective amount of quinolinone derivative of the invention.

While a quinolinone derivative of the invention for use in therapy may be administered in the form of the raw compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the quinolinone derivative of the invention, or a pharmaceutically acceptable salt or derivative thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by the skilled person by use of standard methods and conventional techniques appropriate to the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing from about 0.1 to about 3000 mg of active ingredient per individual dose, preferably from about 1 to about 1000 mg.

The active ingredient may be administered in one or several doses per day.

### Methods of Therapy

The quinolinone derivatives of the present invention are valuable candidates useful for the treatment of a range of ailments involving CNS diseases and disorders, including epilepsy.

In another aspect the invention provides a method for the treatment, prevention or alleviation of a disease or a disorder or a condition of a living animal body, including a human, which disease, disorder or condition is responsive to modulation of CNS, and which method comprises administering to such a living animal body, including a human, in need thereof an effective amount of a quinolinone derivative of the invention.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that suitable dosage ranges are 0.1 to 3000 milligrams daily, dependent as usual upon the exact mode of administration, form in which administered, the indication toward which the administration is directed, the subject involved and the body weight of the subject involved, and further the preference and experience of the physician or veterinarian in charge.

### BRIEF DESCRIPTION OF THE DRAWINGS

The present invention is further illustrated by reference to the accompanying drawing, in which:

FIG. 1 shows the anticonvulsant properties elicited by the Compound 1 (i.e. 2-(6-Bromo-2-oxo-1-quinolyl)acetamide (ED₅₀: 16 mg/Kg).

FIG. 2 shows the antitremogenic properties elicited by compound 1 (i.e. 2-(6-Bromo-2-oxo-1-quinolyl)acetamide reported as a function of time.

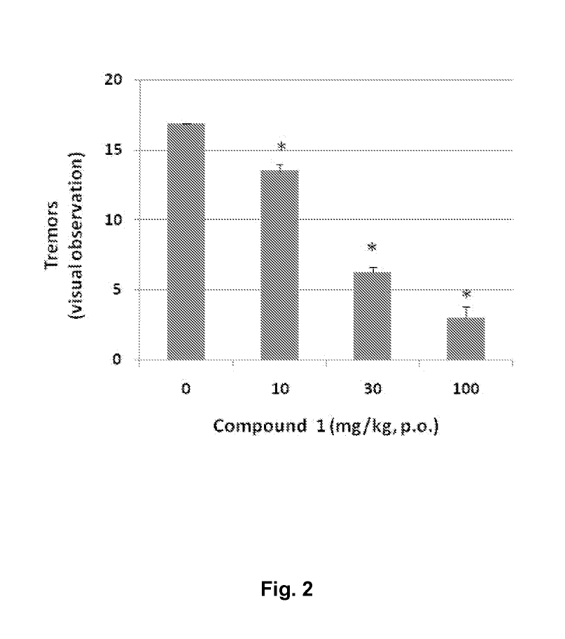

### EXAMPLES

The invention is further illustrated with reference to the following examples, which are not intended to be in any way limiting to the scope of the invention as claimed.

### Example 1

### Preparatory Example

The compounds of the invention were easily prepared by alkylation of commercial or suitably-synthesised benzoquinolones either with 2-bromoacetamide or 2-bromobutanamide in the presence of potassium carbonate in dry N,N-dimethylformamide.

### 2-(6-Bromo-2-oxo-1-quinolyl)acetamide (Compound 1)

A mixture of commercial 6-bromo-2-quinolone (0.50 g, 2.232 mmol), 2-bromoacetamide (0.31 g, 2.232 mmol), potassium carbonate (0.77 g, 5.579 mmol) and N,N-dimethylformamide (10 ml) was heated (70° C.) overnight. The reaction mixture was filtered through a celite bed and this was washed with ethyl acetate. The combined filtrates were evaporated in vacuo and the resulting solid crude product was washed with chloroform, to afford 0.62 g of the title product as an off-white solid (˜100% yield).

LC-ESI−HRMS of [M+H]+ shows 280.9918 Da. Calc. 280.992021Da, dev. −0.8 ppm

M.p. 256.6-257.5° C.

Examples of compounds prepared in a similar manner:

### 2-(6-bromo-2-oxo-1-quinolyl)butanamide (Compound 2)

LC-ESI−HRMS of [M+H]+ shows 309.0236 Da. Calc. 309.023321 Da, dev. 0.9 ppm

M.p. 138.5-140.0° C.

### 2-(8-bromo-2-oxo-1-quinolyl)acetamide (Compound 3)

LC-ESI−HRMS of [M+Na]+ shows 302.9731 Da. Calc. 302.973991 Da, dev. −2.9 ppm

M.p. 158.5-160.0° C.

### 2-(6-chloro-2-oxo-1-quinolyl)acetamide (Compound 4)

LC-ESI−HRMS of [M+Na]+ shows 259.0239 Da. Calc. 259.024506 Da, dev. −2.3 ppm

M.p. 159.4-160.9° C.

### 2-(8-chloro-2-oxo-1-quinolyl)acetamide (Compound 5)

LC-ESI−HRMS of [M+H]+ shows 237.0419 Da. Calc. 237.042536 Da, dev. −2.7 ppm

M.p. 166.5-167.6° C.

### 2-(8-chloro-2-oxo-1-quinolyl)butanamide (Compound 6)

LC-ESI−HRMS of [M+H]+ shows 265.0724 Da. Calc. 265.073836 Da, dev. −5.4 ppm

M.p. 127-128° C.

### 2-(6-chloro-2-oxo-1-quinolyl)butanamide (Compound 7)

LC-ESI−HRMS of [M+Na]+ shows 287.0553 Da. Calc. 287.055806 Da, dev. −1.8 ppm

M.p. 127.5-128.8° C.

### 2-(8-bromo-2-oxo-1-quinolyl)butanamide (Compound 8)

LC-ESI−HRMS of [M+H]+ shows 309.0234 Da. Calc. 309.023321 Da, dev. 0.3 ppm

M.p. 124.2-125.5° C.

### Example 2

### Biological Activity

### Anticonvulsant Properties

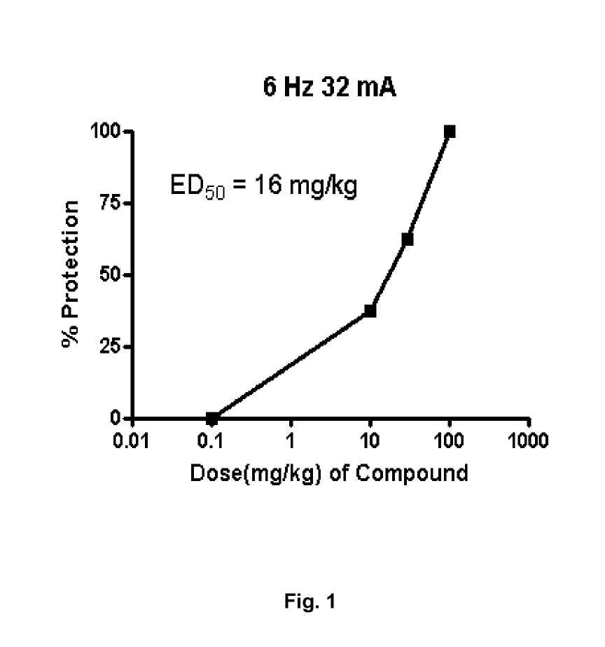

In this example, the anticonvulsant properties elicited by Compound 1 (i.e. 2-(6-Bromo-2-oxo-1-quinolyl)acetamide) (ED₅₀ 16 mg/Kg; pretreatment time 60 mins; p.o.; vehicle: 5% cremaphor) are determined (FIG. 1).

The evaluation of the anticonvulsant properties was carried out in female NMRI mice (20-25 g, Taconic, Denmark). The mice were housed in groups (8 per cage according to weight) with food and water available ad libitum The environment was temperature (20±2° C.) and humidity (55±15%) controlled, and consisted of a 12:12 h light-dark cycle (lights on 06:00 h). The experiments were performed according to the Danish Committee for Experiments on Animals. Psychomotor seizures were induced via corneal stimulation (6 Hz, 0.2 ms rectangular pulses at 32 mA for 3 s) using a Grass S48 stimulator, constant current unit (CCU1) and isolation unit (SIU5), all driven by a stimulus generator (Master-8, AMPI, Israel).

The pads of the corneal stimulator were soaked in 0.9% saline. The mice were restrained during corneal stimulation and released immediately after and observed for the presence or absence of seizure activity. Seizure activity included one of the following behavioral components: stunned, posture awkward but upright, forelimbs often crossed and hindlimbs wide spread, tail is frequently held practically vertically (Straub-tail), stunning may occasionally be preceded by a few seconds of running with a rolling gait, face and forelimb movements resemble “purposeful” automatisms, not infrequently the mouse will stand on almost erect on its hindlimbs while exhibiting automatic behavior, or catatonia is often present.

The duration of any of these seizure variables is 10-75 seconds. In the presence of any of the seizure variables the mice was labeled 0% protection, and if none of the above, the mice was labeled 100% protection.

For screening purposes, the test compound was tested at 1 dose at 1 time-point. In the case of protection of 3 or more out of 8 mice, the compound were further evaluated in dose response (N=8/group). The data were plotted as a function of logarithmic dose and fitted to a sigmoid-dose response (with variable slope (GraphPad Prism, ver. 4) and used to estimate the dose that would give a 50% protection from seizures (N=8/group).

### Antitremorgenic Properties

The antitremorgenic properties elicited by compound 1 (2-(6-Bromo-2-oxo-1-quinolyl)acetamide) is herein described. The evaluation of the antitremorgenic properties was carried out in female NMRI mice (20-25 g, Taconic, Denmark). The mice were housed in groups (8 per cage according to weight) with food and water available ad libitum The environment was temperature (20±2° C.) and humidity (55±15%) controlled, and consisted of a 12:12 h light-dark cycle (lights on 06:00 h). The experiments were performed according to the Danish Committee for Experiments on Animals.

Mice were p.o. injected with compound 1, one hour before the harmaline injection. Tremors were induced by one injection of harmaline (20 mg/kg, s.c.), following the injection mice were observed for 60 minutes and manually scored every 10 minutes using the following rating scale: “0”: No tremors; “1”: mild tremors (occasional muscle twitches or a slight tremor that is barely visible at the head region); “2”: moderate intermittent tremor (intermittent tremor restricted to the head regions); “3”: moderate persistent tremor (visible tremors with occasional quiescent periods, affecting the anterior region); “4”: pronounced severe tremor (continuous severe, gross, whole body tremor). Data data are presented as mean±SEM. *P<0.05, vs. Corresponding vehicle+harmaline group (two way ANOVA followed by Fishers LSD test).

1. A quinolinone derivative represented by Formula I 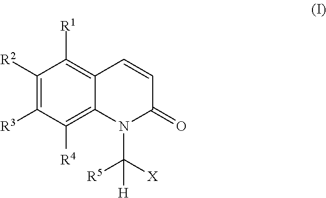
a stereoisomer thereof or a mixture of its stereoisomers, or a pharmaceutically acceptable salt thereof, wherein
R¹, R², R³ and R⁴, independently of each other, represent hydrogen, halo, cyano or SO₂CH₃;
R⁵ represents hydrogen, halo or alkyl; and
X represents an imidazole group, or the group —(CO)N(R⁶, R⁷), wherein
R⁶ and R⁷, independently of each other, represent hydrogen or alkyl.

2. The quinolinone derivative of claim 1, a stereoisomer thereof or a mixture of its stereoisomers, or a pharmaceutically acceptable salt thereof, wherein R¹, R², R³ and R⁴, independently of each other, represent hydrogen, halo, cyano or SO₂CH₃.

3. The quinolinone derivative of claim 1, a stereoisomer thereof or a mixture of its stereoisomers, or a pharmaceutically acceptable salt thereof, wherein R⁵ represents hydrogen, halo or alkyl.

4. The quinolinone derivative of claim 1, a stereoisomer thereof or a mixture of its stereoisomers, or a pharmaceutically acceptable salt thereof, wherein X represents an imidazole group, or the group —(CO)N(R⁶, R⁷), wherein R⁶ and R⁷, independently of each other, represent hydrogen or alkyl.

5. The quinolinone derivative of claim 1, which is
2-(6-Bromo-2-oxo-1-quinolyl)acetamide;
2-(6-bromo-2-oxo-1-quinolyl)butanamide;
2-(8-bromo-2-oxo-1-quinolyl)acetamide;
2-(6-chloro-2-oxo-1-quinolyl)acetamide;
2-(8-chloro-2-oxo-1-quinolyl)acetamide;
2-(8-chloro-2-oxo-1-quinolyl)butanamide;
2-(6-chloro-2-oxo-1-quinolyl)butanamide;
2-(8-bromo-2-oxo-1-quinolyl)butanamide;
a stereoisomer thereof or a mixture of its stereoisomers, or a pharmaceutically acceptable salt thereof.

6. A pharmaceutical composition comprising a therapeutically effective amount of a quinolinone derivative of claim 1, a stereoisomer thereof or a mixture of its stereoisomers, or a pharmaceutically acceptable addition salt thereof, together with at least one pharmaceutically acceptable carrier or diluent.

7-9. (canceled)

10. A method of treatment, prevention or alleviation of a disease or a disorder or a condition of a living animal body, including a human, which disorder, disease or condition is responsive to modulation of CNS, which method comprises the step of administering to such a living animal body in need thereof a therapeutically effective amount of a quinolinone derivative of claim 1, a stereoisomer thereof or a mixture of its stereoisomers, or a pharmaceutically acceptable salt thereof.

11. The method according to claim 10, wherein the disease, disorder or condition responsive is epilepsy, convulsions, tremor, essential tremor, myoclonus, epileptogenesis, anxiety, Parkinson's disease, Huntington's disease, Amyotrophic Lateral Sclerosis, Gilles de la Tourette's syndrome, depression, mania, manic depression, psychosis, schizophrenia, obsessive compulsive disorders (OCD), panic disorders, an eating disorder including anorexia nervosa, bulimia and obesity, narcolepsy, nociception, AIDS-dementia, senile dementia, peripheral neuropathy, autism, dyslexia, tardive dyskinesia, hyperkinesia, post-traumatic syndrome, social phobia, a sleeping disorder, pseudo dementia, Ganser's syndrome, pre-menstrual syndrome, late luteal phase syndrome, chronic fatigue syndrome, mutism, trichotillomania, jet-lag, hypertension, cardiac arrhythmias, a smooth muscle contraction disorder including convulsive disorders, angina pectoris, premature labour, diarrhoea, asthma, premature ejaculation and erectile difficulty, an endocrine system disorder including thyrotoxicosis and pheochromocytoma, a neurodegenerative disorder, including transient anoxia and induced neuro-degeneration, pain, mild, moderate or severe pain, acute pain, chronic pain, pain of recurrent character, neuropathic pain, pain caused by migraine, postoperative pain, phantom limb pain, neuropathic pain, chronic headache, central pain, pain related to diabetic neuropathy, to post therapeutic neuralgia or to peripheral nerve injury, an inflammatory disorder, including an inflammatory skin disorder, acne, rosacea, Crohn's disease, inflammatory bowel disease, ulcerative colitis and diarrhoea, a disorder associated with withdrawal symptoms caused by termination of use of addictive substances, including nicotine withdrawal symptoms, opioid withdrawal symptoms, including heroin, cocaine and morphine, benzodiazepine withdrawal symptoms including benzodiazepine-like drugs and alcohol.
